(19) United States
(12) Patent Application Publication
(10) Pub. No.: US 2021/0346068 A1
(43) Pub. Date: Nov. 11, 2021

(54) TTC Cement Spacer Mold System

(71) Applicant: Brent Austin McNeely, Breinigsville, PA (US)

(72) Inventor: Brent Austin McNeely, Breinigsville, PA (US)

(21) Appl. No.: 17/307,890

(22) Filed: May 4, 2021

Related U.S. Application Data
(63) Continuation-in-part of application No. 29/736,400, filed on May 29, 2020.
(60) Provisional application No. 63/020,158, filed on May 5, 2020.

(51) Int. Cl. A61B 17/72; A61B 17/86

(52) U.S. Cl. CPC A61B 17/7291; A61B 2017/00526; A61B 17/866

(57) ABSTRACT

A medical nail mold suited for casting ankle fusion nails is presented. The medical nail mold contains a mold body, a mold cavity, a mold opening, and a core guide. The mold cavity traverses within the mold body. The mold opening is positioned adjacent to the mold cavity. The core guide is connected adjacent to the mold body. The core guide is aligned with the mold opening and the mold cavity.

The current application claims a priority to the U.S. Provisional Patent application Ser. No. 63/020,158 filed on May 5, 2020.

### FIELD OF THE INVENTION

The present invention relates to cement spacer mold within the medical field. The present invention provides a bone cement spacer mold system to create a bone cement mold for patients in need, such as patients under one-stage or two-stage revision arthroplasty/arthrodesis.

### BACKGROUND OF THE INVENTION

Within the medical fields, bone cement spacer mold can be an issue when it comes to specifics qualities that are needed for patients. There are few known bone cement spacer molds in which exist but seemingly each specific mold can only cover a narrow form or placement of the bone spacer that is created from the molds. One example is the difficulty with centering a Kirschner wire within the bone cement. Many mold spacers are designed horizontally which can cause the bone cement to be uneven which causes uneven centering of the Kirschner wire. Another issue that arises from many of existing bone cement spacer mold is that the accessibility of different sized bone cement created from the molds are limited to the mold itself. Many existing bone cement spacer molds are unable to create different lengths of bone cement as each patient are different from one another and desire different length of bone cement to satisfy their needs.

An objective of the present invention is to provide the user a way of making bone cement molds. The bone cement spacer mold is able to create a variety length of bone cement to satisfy the user's needs for such applications in revising Tibiotalocalcaneal arthrodesis (TTC) and/or other long bone/screws removals and revisions. Also note that the present invention allows the Kirschner wire to be centered along the body of the mold as to other bone cement spacer molds. Furthermore, the present invention provides a mean of long-term and short-term use of the device as there are reusable molds and disposable molds.

### SUMMARY OF THE INVENTION

The present invention is a medical nail mold suited for casting ankle fusion nails. The medical nail mold comprises a mold body, a mold cavity, a mold opening, and a core guide. The mold cavity traverses within the mold body. The mold opening is positioned adjacent to the mold cavity. The core guide is connected adjacent to the mold body. The core guide is aligned with the mold opening and the mold cavity.

### BRIEF DESCRIPTION OF THE DRAWINGS

FIG. 1 is a top perspective view of the present invention.

FIG. 2 is an exploded view of the present invention.

FIG. 3 is a bottom perspective view of the present invention.

FIG. 4 is an exploded view of the present invention, in accordance to another embodiment.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

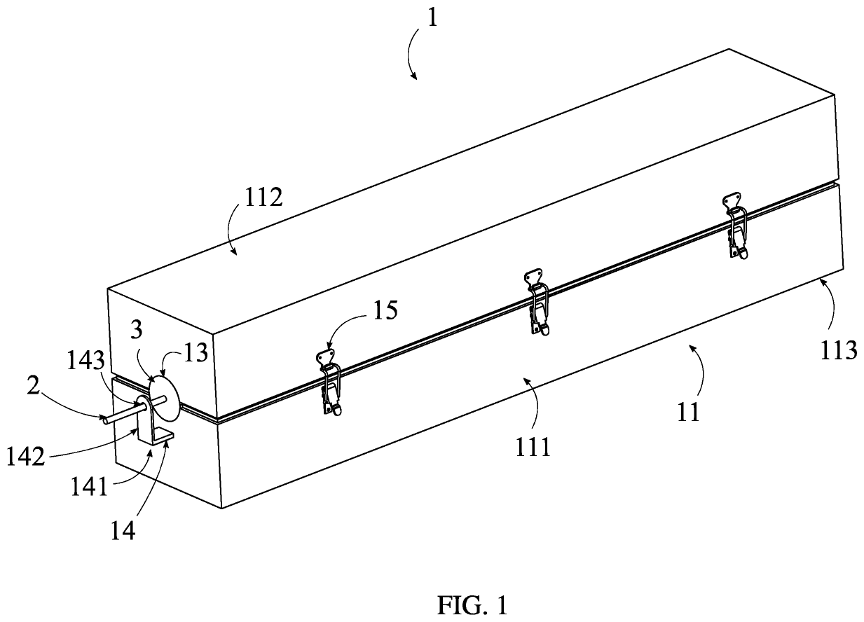

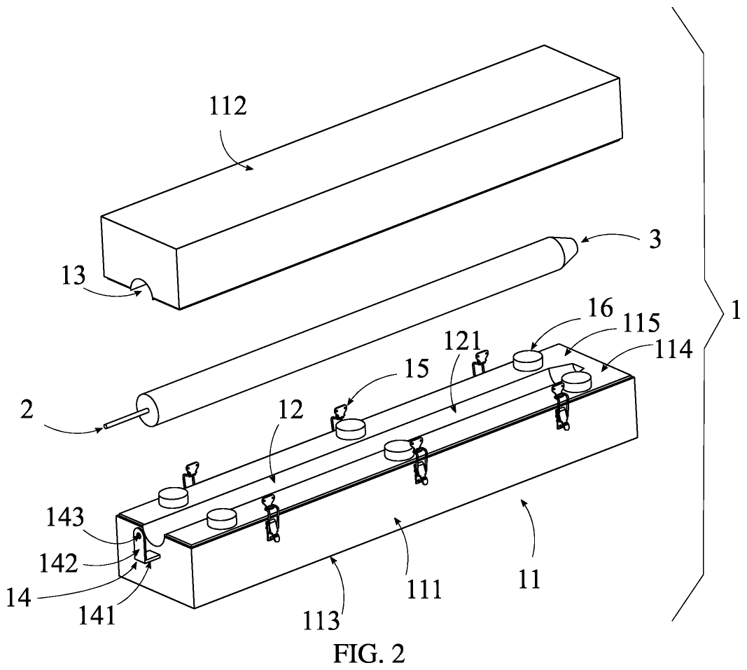

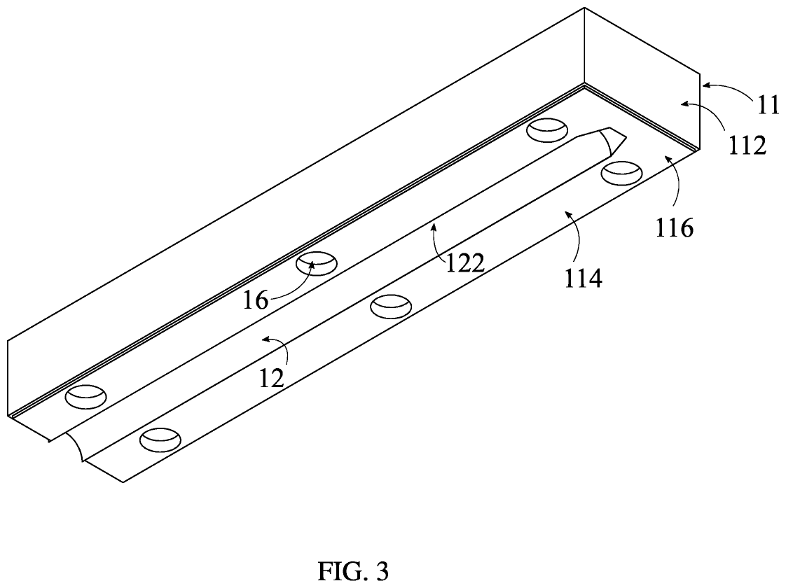

In reference to FIGS. 1-2 and 4, the present invention is a medical nail mold 1 suited for casting a cast medical nail 3. The medical nail mold 1 comprises a mold body 11, a mold cavity 12, a mold opening 13, and a core guide 14. The mold cavity 12 traverses within the mold body 11, as shown in FIGS. 2-3. The mold opening 13 is positioned adjacent to the mold cavity 12, as shown in FIGS. 1-2. The core guide 14 is connected adjacent to the mold body 11, as shown in FIGS. 1-2. The core guide 14 is aligned with the mold opening 13 and the mold cavity 12, as shown in FIGS. 1-2. In the preferred embodiment of the present invention, the medical nail mold 1 is suited for casting a cast medical nail 3, specifically for intermedullary ankle nail fusion operations. The cast medical nail 3 is casted out of a suitable material, such as, but not limited to polymethyl methacrylate (PMMA) material. In the preferred embodiment of the present invention, the mold body 11 is made out of a rigid material suitable for medical nail casting, such as, but not limited to, plaster, aluminum, stainless steel, polymer, or any other suitable material. In the preferred embodiment of the present invention, the core guide 14 allows the installation of a reinforcement wire 2. More specifically, the reinforcement wire 2 may take the form of a medical grade k-wire that provides structural integrity to the PMMA cast medical nail 3.

In the preferred embodiment of the present invention, the mold body 11 comprises a first half 111 and a second half 112, as shown in FIGS. 1-2 and 4. In reference to FIGS. 2-3, the mold cavity 12 comprises a first cavity 121 and a second cavity 122. The first cavity 121 traverses along the first half 111, as shown in FIG. 2. The second cavity 122 traverses along the second half 112, as shown in FIG. 3. The first half 111 is removably attached to the second half 112 through at least one fastening element 15, where the first half 111 and the second half 112 are configured to align the first cavity 121 and the second cavity 122 together to form the mold cavity 12, as shown in FIG. 1. In the preferred embodiment of the present invention, the at least one fastening element 15 is a latch fastener, but may take the form of any other suitable fastener that compresses and holds the first half 111 and the second half 112 together, such as but not limited to screws, ties, clips, or any other suitable fastener.

In reference to FIGS. 2-3, the medical nail mold 1 further comprises at least one alignment implement 16. The at least one alignment implement 16 is distributed about the first half 111 and the second half 112, where each of the at least one alignment is configured to align the first cavity 121 and the second cavity 122 together to form the mold cavity 12. In the preferred embodiment of the present invention, the at least one alignment element is a dowel alignment implement, but may take the form of any other alignment implement, such as, but not limited to alignment pins, magnets, brackets, or any other suitable alignment implement that aligns the first half 111 and the second half 112 together.

In reference to FIG. 2, the mold body 11 further comprises a mold housing 113 and at least one mold insert 114. The at least one mold insert 114 is connected within the mold housing 113. The mold cavity 12 is positioned within the at least one mold insert 114. The at least one mold insert 114 is made out of a silicone material or any other suitable casting mold material. In the preferred embodiment of the present invention, the mold housing 113 is made out of a rigid material, such as, but not limited to stainless steel, aluminum, polymer, or any other suitable material. In reference to FIGS. 2-3, the at least one mold insert 114 comprises a first insert 115 and a second insert 116. The first insert 115 is connected within the first half 111. The second insert 116 is connected within the second half 112. The first cavity 121 traverses along the first insert 115. The second cavity 122 traverses along the second insert 116. The first half 111 is removably attached to the second half 112 through at least one fastening element 15, where the first half 111 and the second half 112 are configured to align the first insert 115 and the second insert 116 together to form the mold cavity 12.

In reference to FIGS. 1-2, the core guide 14 comprises a first flange 141, a second flange 142, and a guide aperture 143. The first flange 141 is connected adjacent to the mold body 11. The second flange 142 is perpendicularly positioned to the first flange 141. The guide aperture 143 traverses through the second flange 142. The guide aperture 143 is concentrically aligned with the mold opening 13 and the mold cavity 12. In the preferred embodiment of the present invention, the core guide 14 may take the form of an angle iron bracket that aligns the reinforcement wire 2 along the mold opening 13 and the mold cavity 12.

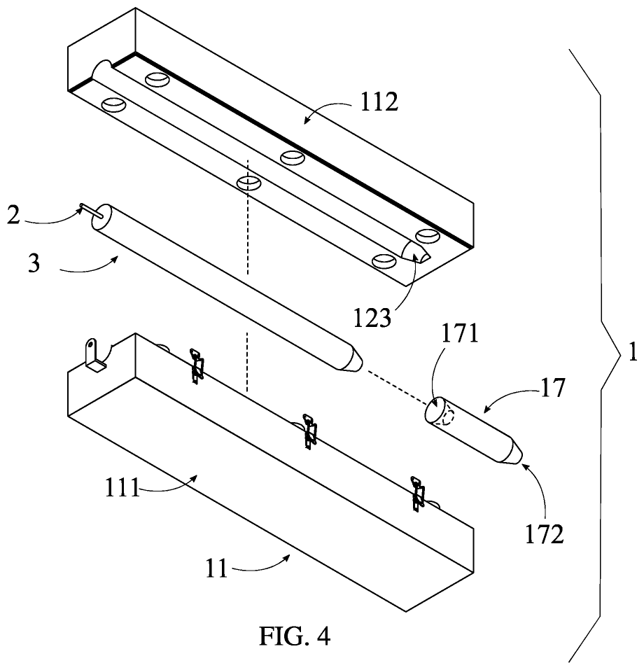

In another embodiment, the medical nail mold 1 further comprises a spacer insert 17, as shown in FIG. 4. The mold cavity 12 comprises a first tip cavity 123. The spacer insert 17 comprises a second tip cavity 171 and a tip end 172. The first tip cavity 123 and the mold opening 13 are positioned terminally opposite to each other along the mold cavity 12. The tip end 172 and the second tip cavity 171 are positioned terminally opposite to each other along the spacer insert 17, where the base is configured to take form of the first tip cavity 123. The tip end 172 is positioned adjacent to the first tip cavity 123. In various embodiments, the spacer insert 17 may vary in length, such that the medical nail mold 1 forms a shortened length from the original mold cavity 12 length profile.

Although the invention has been explained in relation to its preferred embodiment, it is to be understood that many other possible modifications and variations can be made without departing from the spirit and scope of the invention as hereinafter claimed.

What is claimed is:

1. A medical nail mold comprising:
a mold body;
a mold cavity;
a mold opening;
a core guide;
the mold cavity traversing within the mold body;
the mold opening being positioned adjacent to the mold cavity;
the core guide being connected adjacent to the mold body; and
the core guide being aligned with the mold opening and the mold cavity.

2. The medical nail mold as claimed in claim 1 comprising:
the mold body comprising a first half and a second half;
the mold cavity comprising a first cavity and a second cavity;
the first cavity traversing along the first half;
the second cavity traversing along the second half; and
the first half being removably attached to the second half through at least one fastening element, wherein the first half and the second half are configured to align the first cavity and the second cavity together to form the mold cavity.

3. The medical nail mold as claimed in claim 2 comprising:
at least one alignment implement; and
the at least one alignment implement being distributed about the first half and the second half, wherein each of the at least one alignment is configured to align the first cavity and the second cavity together to form the mold cavity.

4. The medical nail mold as claimed in claim 1 comprising:
the mold body comprising a mold housing and at least one mold insert;
the at least one mold insert being connected within the mold housing; and
the mold cavity being positioned within the at least one mold insert.

5. The medical nail mold as claimed in claim 4 comprising:
the mold body comprising a first half and a second half;
the at least one mold insert comprising a first insert and a second insert;
the mold cavity comprising a first cavity and a second cavity;
the first insert being connected within the first half;
the second insert being connected within the second half;
the first cavity traversing along the first insert;
the second cavity traversing along the second insert; and
the first half being removably attached to the second half through at least one fastening element, wherein the first half and the second half are configured to align the first insert and the second insert together to form the mold cavity.

6. The medical nail mold as claimed in claim 1 comprising:
the core guide comprising a first flange, a second flange, and a guide aperture;
the first flange being connected adjacent to the mold body;
the second flange being perpendicularly positioned to the first flange;
the guide aperture traversing through the second flange; and
the guide aperture being concentrically aligned with the mold opening and the mold cavity.

7. The medical nail mold as claimed in claim 1 comprising:
a spacer insert;
the mold cavity comprising a first tip cavity;
the spacer insert comprising a second tip cavity and a tip end;
the first tip cavity and the mold opening being positioned terminally opposite to each other along the mold cavity;
the tip end and the second tip cavity being positioned terminally opposite to each other along the spacer insert, wherein the base is configured to take form of the first tip cavity; and
the tip end being positioned adjacent to the first tip cavity.

8. The medical nail mold as claimed in claim 2, wherein the at least one fastening element is a latch fastener.

9. The medical nail mold as claimed in claim 3, wherein the at least one alignment element is a dowel alignment implement.

10. The medical nail mold as claimed in claims 4 and 5, wherein the at least one mold insert is made out of a silicone material.

11. The medical nail mold as claimed in claims 4 and 5, wherein the mold housing is made out of a rigid material.
